(19) United States
(12) Patent Application Publication
(10) Pub. No.: US 2009/0247537 A1
(43) Pub. Date: Oct. 1, 2009

(54) METHODS FOR PREVENTING OR TREATING BRUXISM USING DOPAMINERGIC AGENTS

(76) Inventor: William Dale Overfield, Edgewood, WA (US)

Correspondence Address: BLACK LOWE & GRAHAM, PLLC, 701 FIFTH AVENUE, SUITE 4800, SEATTLE, WA, 98104, US

(21) Appl. No.: 12/264,806

(22) Filed: Nov. 4, 2008

Related U.S. Application Data
(63) Continuation-in-part of application No. 12/054,816, filed on Mar. 25, 2008, now abandoned.

(51) Int. Cl. A61K 31/506; A61K 31/428; A61K 31/381; A61K 31/136; A61K 31/4045; A61K 31/437

(52) U.S. Cl. 514/252.14; 514/367; 514/438; 514/415; 514/292; 514/657

(57) ABSTRACT

Methods for preventing or treating bruxism, including alleviating or eliminating one or more symptoms, diseases or conditions associated with or resulting from bruxism, using dopaminergic agents such as monoamine oxidase inhibitors that increase dopaminergic activity and dopamine agonists, are disclosed.

### REFERENCE TO RELATED APPLICATIONS

This application is a continuation-in-part of U.S. patent application Ser. No. 12/054,816 filed on Mar. 25, 2008, which is incorporated herein by reference in its entirety.

### FIELD OF THE INVENTION

The present invention relates to novel methods for preventing or treating bruxism, including alleviating or eliminating one or more symptoms, diseases or conditions associated with or resulting from bruxism, using dopaminergic agents such as monoamine oxidase inhibitors that increase dopaminergic activity and dopamine agonists.

### BACKGROUND OF THE INVENTION

Bruxism, otherwise known as teeth clenching or teeth grinding, is a commonly occurring condition involving forceful contact between the biting surfaces of the upper and lower teeth. Bruxism is typically accompanied by clenching of the jaw. Bruxism often occurs during sleep, although it can also occur while an individual is awake. While the causes of bruxism are unknown, factors that have been related to the condition include disturbed sleep patterns, high levels of anxiety and stress, use of serotonin selective reuptake inhibitors, use of the drug Provigil®, consumption of amphetamines and related drugs, and disorders such as Huntington's disease and Parkinson's disease. Bruxism can result in damage to the teeth, including abfractions, abnormal wear of occlusal surfaces, fractures, occlusal trauma and tooth loss. Other symptoms, diseases and conditions resulting from or associated with bruxism include myofacial muscle pain, chronic headaches, migraine headaches, periodontal disease, upper neck pain and temporomandibular disorder.

A number of different methods have been tried for the treatment of bruxism, including psychotherapy, biofeedback, negative feedback techniques, exercise of the mandible, drugs and equilibration adjustment. These methods have been largely ineffective. The most common approach for treating bruxism is splint therapy, which involves the use of splints such as mouthguards, removable appliances and the like. While the use of splints can prevent damage to teeth caused by grinding, this approach does not actually prevent or cure bruxism. Additionally, the use of splints in some cases can cause damage to the teeth, including caries, gum inflammation and splint-induced open bite.

In view of the foregoing, there remains a need for new methods for the prevention and treatment of bruxism, including the reduction or elimination of one or more symptoms, diseases or conditions associated with or resulting from bruxism.

### SUMMARY OF THE INVENTION

It is therefore an object of the present invention to provide new methods for the prevention and treatment of bruxism, including the alleviation or elimination of one or more symptoms, diseases or conditions associated with or resulting from bruxism.

The invention achieves these objects and satisfies additional objects and advantages by providing methods for preventing or treating bruxism, comprising administering to a patient in need of such prevention or treatment a therapeutically effective amount of a dopaminergic agent.

In certain embodiments, the present invention provides methods for preventing or treating bruxism, comprising administering to a patient in need of such prevention or treatment a therapeutically effective amount of a dopamine agonist or a pharmaceutically acceptable salt, enantiomer, solvate, hydrate, polymorph or prodrug thereof

In other embodiments, the present invention provides methods for preventing or treating bruxism, comprising administering to a patient in need of such prevention or treatment a therapeutically effective amount of a monoamine oxidase inhibitor that increases dopaminergic activity or a pharmaceutically acceptable salt, enantiomer, solvate, hydrate, polymorph or prodrug thereof.

In additional embodiments, the present invention provides methods for preventing or treating bruxism wherein such prevention or treatment alleviates or eliminates one or more symptoms, diseases or conditions associated with or resulting from bruxism.

The foregoing objects and additional objects, features, aspects and advantages of the present invention are further exemplified and described in the following detailed description.

### DETAILED DESCRIPTION OF EXEMPLARY EMBODIMENTS OF THE INVENTION

It has been found that compounds that act as dopaminergic agents can be effectively used in the prevention and treatment of bruxism and thereby alleviate or eliminate one or more symptoms, diseases or conditions associated with or resulting from bruxism. This is a surprising result, since some compounds that act as dopaminergic agents, such as amphetamines and L-dopa, have been causally related to bruxism, while another, the ergot derivative bromocriptine, has been shown to be ineffective in reducing the frequency and severity of bruxism during sleep in a double blind, crossover, placebo-controlled clinical trial, thereby disproving earlier preliminary results to the contrary.

The present invention therefore provides methods for preventing or treating bruxism, comprising administering to a patient in need of such prevention or treatment a therapeutically effective amount of a compound that acts as a dopaminergic agent.

As used herein, the term “dopaminergic agent” means a compound or composition that stimulates neural signaling via the dopaminergic system.

Various compounds that act as dopaminergic agents can be used in practicing the methods of the present invention for preventing or treating bruxism. Suitable compounds include, for example, monoamine oxidase inhibitors that increase dopaminergic activity and dopamine agonists. As used herein, the term “dopamine agonist” means a compound that binds to one or more of the different types and subtypes of dopamine receptors and stimulates neural signaling via the dopaminergic system. In certain embodiments, the dopamine agonist preferentially binds particular members of the dopamine receptor family. In additional embodiments, the dopamine agonist preferentially binds one or more members of the D₁-like family of dopamine receptors. In other embodiments, the dopamine agonist preferentially binds one or more members of the D₂-like family of dopamine receptors. In further embodiments, the dopamine agonist preferentially binds the dopamine D₁ receptor subtype. In additional embodiments, the dopamine agonist preferentially binds the dopamine D₂ receptor subtype. In other embodiments, the dopamine agonist preferentially binds the dopamine D₃ receptor subtype. In further embodiments, the dopamine agonist preferentially binds the dopamine D₄ receptor subtype. In additional embodiments, the dopamine agonist preferentially binds the dopamine D₅ receptor subtype. In other embodiments, the dopamine agonist binds multiple dopamine receptor types and subtypes. In further embodiments, the dopamine receptor agonist binds the D₂, D₃ and D₄ dopamine receptor subtypes. In additional embodiments, the dopamine receptor agonist binds the D₂ and D₃ dopamine receptor subtypes. In other embodiments, the dopamine receptor agonist binds the D₁, D₂ and D₃ dopamine receptor subtypes. Dopamine agonists are sometime classified as ergot derivatives (e.g., pergolide, lysuride, cabergoline) and nonergot derivatives such as ropinirole, pramipexole, rotigotine, pardoprunox, piribedil and sumanirole. Suitable dopamine agonists that can be used in practicing the methods of the present invention for preventing or treating bruxism include, for example, pramipexole, ropinirole, rotigotine, pardoprunox, piribedil and sumanirole. Other suitable dopamine agonists include, for example, apomorphine, amantadine, fenoldopam, talipexale and quinpirole. Suitable monoamine oxidase inhibitors that increase dopaminergic activity that can be used in practicing the methods of the present invention include, for example, rasagiline and selegiline.

The dopamine agonist pramipexzole is commercially available and sold under the trademark Mirapex® as the dihydrochloride monohydrate. The chemical name of this compound is (S)-2-amino-4,5,6,7,-tetrahydro-6-(propylamino)benzothiazole dihydrochloride monohydrate. Pramipexzole has been shown to bind the D₂, D₃ and D₄ dopamine receptor subtypes. It is available as tablets for oral administration containing 0.125 mg, 0.25 mg, 0.5 mg, 0.75 mg, 1.0 mg and 1.5 mg of the active compound. The tablets also contain various inactive ingredients including mannitol, corn starch, collodial silicon dioxide, povidone and magnesium stearate. Methods for the preparation of pramipexzole and related compounds and compositions are known in the art and described, for example, in US Pat. Nos. 4,886,812, 6,001,861 and 6,194,445.

The dopamine agonist ropinirole is commercially available and sold under the trademark Requip® as the monohydrochloride salt. The chemical name of this compound is 4-[2-(dipropylamino)ethyl]-1,3-dihydro-2H-indole-2-one monohydrochloride. Ropinirole has been shown to bind the D₂ and D₃ dopamine receptor subtypes. It is available as tablets for oral administration containing 0.25 mg, 0.5 mg, 0.75 mg, 1.0 mg, 2.0 mg, 3.0 mg, 4.0 mg and 5.0 mg of the active compound. The tablets also contain various inactive ingredients including croscarmellose sodium, hydrous lactose, microcrystalline cellulose, magnesium stearate and one or more of the following substances: carmine, FD&C Blue No. 2 aluminum lake, FD&C Yellow No. 6 aluminum lake, hypromellose, iron oxides, polyethylene glycol, polysorbate 80 and titanium dioxide. Methods for the preparation of ropinirole and related compounds and compositions are known in the art and described, for example, in U.S. Pat. Nos. 4,452,808 and 4,824,860.

The dopamine agonist rotigotine is commercially available and sold under the trademark Neupro® as the (6S) enantiomer. The chemical name of this compound is (6S)-6-{propyl[2-(2-thienyl)ethyl]amino}-5,6,7,8-tetrahydro-1-napthalenol. Rotigotine has been shown to bind the D₁, D₂ and D₃ dopamine receptor subtypes. It is available in a transdermal delivery system containing 4.5 mg, 9.0 mg or 13.5 mg of the active compound and capable of delivering 2.0 mg, 4.0 mg or 6.0 mg, respectively, of the active compound over a 24 hour period. The transdermal delivery system also contains various inactive ingredients including ascorbyl palmitate, povidone, silicon adhesive, sodium metabisulfite and dl-alpha-tocopherol. Methods for the preparation of rotigotine and related compounds and compositions are known in the art and described, for example, in U.S. Pat. No. 6,884,434.

The dopamine agonist pardoprunox is currently in clinical trials as the hydrochloride salt. The chemical name of this compound is 7-(4-methylpiperazin-1-yl)-1,3-benzoxazol-(3H)-one hydrochloride. Pardoprunox has been shown to bind the D₂ and D₃ dopamine receptor subtypes as well as well as the 5-HT_{1A} serotonin receptor subtype. It is supplied for clinical trial purposes as tablets for oral administration containing 6 mg and 12 mg of the active compound along with various inactive ingredients. Methods for the preparation of pardoprunox and related compounds and compositions are known in the art and described, for example, in U.S. Pat. No. 4,782,061.

The dopamine agonist sumanirole is currently in clinical trials as the maleate salt. The chemical name of this compound is (R)-5,6-dihydro-5-(methylamino)-4H-imidazol[4,5,1-ij]quinolin-2(1H)-one(Z)-2-butenedioate. Sumanirole has been shown to selectively bind the D₂ dopamine receptor subtype. It is available for clinical trial purposes as tablets for oral administration containing 0.5 mg, 1 mg, 2 mg, 4 mg, 8 mg and 24 mg of the active compound. The tablets also contain various inactive ingredients including a matrix comprising hydroxypropylmethylcellulose (HPMC) and starch. Methods for the preparation of sumanirole and related compounds and compositions are known in the art and described, for example, in U.S. Pat. Nos. 5,273,975 and 5,436,240.

The dopamine agonist piribedil is commercially available and sold under the trademark Trivastal® as the maleate salt. The chemical name of this compound is 2-[4-(benzo[1,3]dioxol-5-ylmethyl)piperazin-1-yl]pyrimidine. Piribedil has been shown to bind the D₂ and D₃ dopamine receptor subtypes as well as the α₂ₐ and α_{2c} adrenergic receptor subtypes. It is available as tablets for oral administration containing 50 mg of the active compound. The tablets also contain various inactive ingredients including starch, collodial silicon dioxide, stearic acid and talc. Methods for the preparation of piribedil and related compounds and compositions are known in the art and described, for example, in U.S. Pat. No. 3,299,067.

The monoamine oxidase inhibitor rasigiline is commercially available and sold under the trademark Azilect® as the mesylate salt. The chemical name of this compound is (1R)-N-prop-2-ynyl-2,3-dihydro-1H-inden-1-amine methanesulfonate. It acts as a selective inhibitor of MAO-B. It is available as tablets for oral administration containing an amount of rasigiline mesylate equivalent to 0.5 mg or 1.0 of rasigiline base. The tablets also contain various inactive ingredients including mannitol, starch, collodial silicon dioxide, stearic acid and talc. Methods for the preparation of rasigiline and related compounds and compositions are known in the art and described, for example, in U.S. Pat. Nos. 5,387,612, 5,454,446, 5,457,133, 5,532,415, 5,786,390 and 6,126,968.

The monoamine oxidase inhibitor selegiline is commercially available and sold under the trademark Emsam®. The chemical name of this compound is (−)-(N)-methyl-N-[(1R)-1-methyl-2-phenylethyl]prop-2-yn-1-amine. It acts as an inhibitor of both MAO-A and MAO-B. It is available in a transdermal delivery system containing 1.0 mg/cm² of the active compound in sizes of 20 mg/cm², 30 mg/cm² and 40 mg/cm² and capable of delivering 6.0 mg, 9.0 mg or 12 mg, respectively, of the active compound over a 24 hour period. The transdermal delivery system also contains various inactive ingredients including acrylic adhesive, ethylene vinyl acetate/polyethylene polyester, polyurethane and silicon coated polyester. Methods for the preparation of selegiline and related compounds and compositions are well-known in the art and described, for example, in U.S. Pat. Nos. 5,423,342, 6,423,342, 7,070,808, 7,150,881 and RE34,579.

In general, the compounds that can be used in practicing the methods of the present invention can be provided in a variety of forms, including pharmaceutically acceptable, active salts, solvates, hydrates, polymorphs, and/or prodrugs of the compounds disclosed herein, or any combination thereof. The compounds that can be used in practicing the methods of the present invention can also be provided as enantiomers, diastereomers, and other stereoisomeric forms, including racemic and resolved forms and mixtures thereof. The individual enantiomers may be separated according to methods that are well known to those of ordinary skill in the art. Also contemplated are derivatives and modifications of the compounds disclosed hereunder.

As used herein, the term “prodrug” refers to any covalently bonded carriers which release the active parent drug in vivo. Examples of prodrugs include esters or amides of a compound that can be used in practicing the methods of the present invention with hydroxyalkyl or aminoalkyl as a substituent. These may be prepared by reacting such compounds with anhydrides such as succinic anhydride.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule.

The compounds that can be used in practicing the methods of the present invention can be can be prepared as both acid addition salts formed from an acid and base salts. Suitable acid addition salts include, for example, hydrochloride, hydrobromide, hydroiodide, sulphate, hydrogen sulphate, nitrate, phosphate, and hydrogen phosphate salts. Other examples of pharmaceutically acceptable acid addition salts include inorganic and organic acid addition salts. Additional pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as mesylate, benzenesulfonate, p-toluenesulfonate and the like; and amino acid salts such as arginate, asparginate, glutamate, tartrate, gluconate and the like. Suitable base salts are formed from bases, which form non-toxic salts and include, for example, aluminum, calcium, lithium, magnesium, potassium, sodium, zinc and diethanolamine salts.

In accordance with the present invention, the compounds that can be used in practicing the methods of the present invention, optionally formulated with additional ingredients in a pharmaceutically acceptable composition, are administered to mammalian subjects, for example a human patient, to prevent and treat bruxism and thereby alleviate or eliminate one or more symptoms, diseases or conditions associated with or resulting from bruxism. Symptoms, diseases or conditions that can be alleviated or eliminated include, for example, chronic headache such as chronic daily headache, tooth damage, migraine headache, upper neck pain, periodontal disease and temporomandibular disorder, jaw pain, atypical face pain and myofacial pain. In certain embodiments, “treatment” or “treating” refers to amelioration of bruxism or one or more symptoms of bruxism, whereby the symptom(s) is/are alleviated by increasing dopaminergic activity. In other embodiments, “treatment” or “treating” refers to an amelioration of at least one measurable physical parameter associated with bruxism. In yet another embodiment, “treatment” or “treating” refers to inhibiting or reducing the progression or severity of bruxism (or one or more symptoms thereof), e.g., as discerned based on physical, physiological, and/or psychological parameters. In additional embodiments, “treatment” or “treating” refers to delaying the onset of bruxism (or one or more symptoms thereof).

In certain embodiments, the compounds that can be used in practicing the methods of the present invention are administered to a mammalian subject, for example a human patient, as a preventative or prophylactic treatment against bruxism (or one or more symptom(s) thereof). As used herein, “prevention”, “preventing”, and prophylaxis refers to a reduction in the risk or likelihood that the subject will acquire bruxism or one or more symptoms thereof. Alternatively, prevention and prophylaxis may correlate with a reduced risk of recurrence of bruxism in the subject once the subject has been cured, restored to a normal state, or placed in remission from bruxism.

Administration of an effective amount of a compound that can be used in practicing the methods of the present invention to a mammalian subject presenting with bruxism or one or more symptoms, diseases or conditions associated with or resulting from bruxism will detectably alleviate, eliminate, or prevent bruxism and/or one or more of the associated or resultant symptoms, diseases or conditions. In exemplary embodiments, administration a compound that can be used in practicing the methods of the present invention to a mammalian subject will detectably alleviate, eliminate, or prevent bruxism or one or more symptoms, diseases or conditions associated with or resulting from bruxism by at least 10%, 20%, 30%, 50% or greater, up to a 75-90%, or 95% or greater.

An “effective amount,” “therapeutic amount,” “therapeutically effective amount,” or “effective dose” is an effective amount or dose of a compound that can be used in practicing the methods of the present invention sufficient to elicit a desired pharmacological or therapeutic effect in a mammalian subject—typically resulting in a measurable reduction in the occurrence, frequency, or severity of bruxism, including any combination of symptoms, diseases, or conditions, associated with or caused by bruxism, in the subject. In certain embodiments, when a compound that can be used in practicing the methods of the present invention is administered to treat bruxism, an effective amount of the compound will be an amount sufficient in vivo to delay or eliminate onset of the symptoms of bruxism. Therapeutic efficacy can alternatively be demonstrated by a decrease in the frequency or severity of symptoms associated bruxism, or by altering the nature, recurrence, or duration of symptoms associated with bruxism. Therapeutically effective amounts, and dosage regimens, of a compound that can be used in practicing the methods of the present invention, including pharmaceutically effective salts, solvates, hydrates, polymorphs or prodrugs thereof, will be readily determinable by those of ordinary skill in the art, often based on routine clinical or patient-specific factors.

Suitable routes of administration and delivery methods for a compound that can be used in practicing the methods of the present invention include, but are not limited to, oral, buccal, nasal, aerosol, topical, transdermal, mucosal, rectal, parental, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection and infusion technology.

Suitable effective unit dosage amounts of a compound that can be used in practicing the methods of the present invention for mammalian subjects may range from about 0.01 mg to about 100 mg, about 0.125 mg to about 100 mg, about 0.25 mg to about 50 mg, about 0.5 mg to about 25 mg or about 1.0 mg to about 12 mg. In other embodiments, the effective dosage will be selected within narrower ranges of, for example, about 0.125 mg to about 12 mg, about 12 mg to about 25 mg, about 25 mg to about 50 mg and about 50 mg to about 100 mg. In additional embodiments, unit dosage amounts of a compound that can be used in practicing the methods of the present invention for mammalian subjects may range from about 0.125 mg to about 1.5 mg for pramipexzole, about 0.25 mg to about 5.0 mg for ropinirole, about 0.5 mg to about 6.0 mg for rotigotine, about 0.5 mg to about 1.0 mg for rasigiline, about 6.0 mg to about 12 mg for selegiline, about 6 mg to about 42 mg for pardoprunox, about 0.5 mg to about 8 mg for sumanirole and about 50 mg to about 300 mg for piribedil. These and other effective unit dosage amounts may be administered in a single dose, or in the form of multiple daily, weekly or monthly doses, for example in a dosing regimen comprising from 1 to 5, or 2 to 3, doses administered per day, per week, or per month. In certain embodiments, dosages of about 0.01 mg to about 100 mg, about 0.125 mg to about 100 mg, about 0.25 mg to about 50 mg, about 0.5 mg to about 25 mg or about 1.0 mg to about 12 mg are administered one, two, three, or four times per day. In other embodiments, dosages of about 0.125 mg to about 12 mg, about 12 mg to about 25 mg, about 25 mg to about 50 mg and about 50 mg to about 100 mg are administered one, two or three times per day. In additional embodiments, pramipexzole is administered in dosages from about 0.125 mg to about 1.5 mg three times per day, ropinirole is administered in dosages from about 0.25 mg to about 5.0 mg three times per day, rotigotine is administered in dosages of about 0.5 mg to about 6.0 mg once per day, rasigiline is administered in dosages of about 0.5 mg to about 1.0 mg once per day, selegiline is administered in dosages of about 6.0 mg to about 12 mg once per day, pardoprunox is administered in dosages of about 6 mg to about 42 mg per day, sumanirole is administered in dosages of about 0.5 mg to about 8 mg per day and piribedil is administered in dosages of about 50 mg to about 300 mg per day. In alternate embodiments, dosages are calculated according to body weight, based on the dosage criteria set forth above.

The amount, timing and mode of delivery of compounds that can be used in practicing the methods of the present invention will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds that can be used in practicing the methods of the present invention will ordinarily be selected to approximate a minimal dosing regimen that is necessary and sufficient to substantially treat or prevent bruxism or alleviate or eliminate one or more symptoms, diseases or conditions associated with or resulting from bruxism in the subject, as described herein.

It should be understood that the compounds that can be used in practicing the methods of the present invention can be used in combination with other drugs (e.g., by co-administration, concurrent administration or sequential administration) that provide additional therapeutic benefits. For example, the compounds that can be used in practicing the methods of the present invention can be used in combination with other drugs that treat symptoms, diseases, or conditions associated with or caused by bruxism, including chronic headache, tooth damage, migraine headache, upper neck pain, periodontal disease and temporomandibular disorder. Examples of drugs providing symptomatic relief for chronic headache, migraine headache, upper neck pain and temporomandibular disorder include: non-sterodial anti-inflammatory drugs (NSAID's) such as ketoprofen, aminopyrine, amodiaquine, ampyrone, antipyrine, apazone, aspirin, benzydamine, bromelains, bufexamac, clofazimine, clonixin, curcumin, dapsone, diclofenac, diflunisal, dipyrone, epirizole, etodolac, fenoprofen, flufenamic acid, flurbiprofen, glycyrrhizic acid, ibuprofen, indomethacin, ketorolac, ketorolac tromethamine, meclofenamic acid, mefenamic acid, mesalamine, naproxen, niflumic acid, oxyphenbutazone, pentosan sulfuric polyester, phenylbutazone, piroxicam, prenazone, salicylates, sodium salicylate, sulfasalazine, sulindac, suprofen, and tolmetin; and analgesics/antipyretics such as ketoprofen, flurbiprofen, aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride and meprobamate. Other examples of drugs providing symptomatic relief from migraine headache include triptans, ergotamine tartrate, propanolol hydrochloride, isometheptene mucate and dichloralphenazone.

In general, compounds that can be used in practicing the methods of the present invention can be formulated into pharmaceutical compositions for administration to an individual according to standard pharmaceutical texts and protocols (e.g., Remington's Pharmaceutical, 18^{th} ed., Alfonso R. Gennaro, ed. (Mack Publishing Co., Easton, Pa. 1990)). Such pharmaceutical compositions may optionally include excipients recognized in the art of pharmaceutical compounding as being suitable for the preparation of dosage units as described herein. Such excipients include, without limitation, binders, fillers, lubricants, emulsifiers, suspending agents, sweeteners, flavorings, preservatives, buffers, wetting agents, disintegrants, effervescent agents and other conventional excipients and additives.

The compositions containing compounds that can be used in practicing the methods of the present invention can thus include any one or combination of the following: one or more pharmaceutically acceptable carriers or excipients; other medicinal agent(s); pharmaceutical agent(s); adjuvants; and various other pharmaceutical additives and agents known to those skilled in the art. These additional formulation additives and agents will often be biologically inactive and can be administered to patients without causing unacceptable deleterious side effects or serious adverse interactions with the active agent.

Compositions containing compounds that can be used in practicing the methods of the present invention will most often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, capsules, caplets, pills, lozenges, powders, granuels, solutions, suspensions, syrups or elixirs which may be prepared by any conventional method of preparing pharmaceutical oral unit dosage form. Oral unit dosage forms, such as tablets, may contain one or more conventional additional formulation ingredients, including, but not limited to, release modifying agents, glidants, compression aides, effervescent agents, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate.

If desired, the compounds that can be used in practicing the methods of the present invention can be administered in a controlled release form, for example by use of a slow release carrier such as a hydrophilic, slow release polymer. Exemplary controlled release agents in this context include, but are not limited to, hydroxypropyl methyl cellulose, having a viscosity in the range of about 100 cps to about 100,000 cps.

Yet additional compositions and methods of the invention are provided for topical administration of compounds that can be used in practicing the methods of the present invention. Topical compositions may comprise a compound that can be used in practicing the methods of the present invention and any other active or inactive component(s) incorporated in a dermatological or mucosally acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound that can be used in practicing the methods of the present invention dissolved or dispersed in a portion of a water or other solvent or liquid to be incorporated in the topical composition or delivery device. Transdermal administration may be enhanced by the use of dermal penetration enhancers known to those skilled in the art.

Yet additional formulations are provided for parenteral administration of compounds that can be used in practicing the methods of the present invention, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; and aqueous and non-aqueous dispersions and emulsions. The formulations may be presented in unit-dose or multi-dose containers.

Compositions comprising compounds that can be used in practicing the methods of the present invention may also include polymers for extended release following parenteral administration. Such polymeric materials are well known to those of ordinary skill in the pharmaceutical compounding arts. Pharmaceutically acceptable formulations and ingredients will typically be sterile or readily sterilizable, biologically inert, and easily administered. Parenteral preparations typically contain buffering agents and preservatives, and may be lyophilized to be re-constituted at the time of administration. Injection solutions, dispersions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, compositions comprising compounds that can be used in practicing the methods of the present invention may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macro emulsions.

Pharmaceutical compositions comprising compounds that can be used in practicing the methods of the present invention may be packaged in a variety of containers appropriate to the dosage form and mode of administration. These include but are not limited to vials, bottles, cans, packets, ampoules, cartons and flexible containers.

The invention also provides pharmaceutical packs or kits comprising one or more containers holding a composition comprising a compound that can be used in practicing the methods of the present invention as described herein, including pharmaceutically acceptable salts and other forms of such compounds, in a pharmaceutically acceptable, stable form. Optionally packaged with these packs and kits can be a notice, e.g., in a form prescribed by a governmental agency regulating pharmaceuticals or biological products, reflecting approval by the agency of the manufacture, use and/or sale of the product contained in the pack or kit for human administration (optionally specifying one or more approved treatment indications as described herein).

The following examples illustrate certain embodiments of the present invention, and are not to be construed as limiting the present disclosure.

### EXAMPLES

### Example 1

Nine patients with symptoms and evidence of bruxism were treated with pramipexzole (Mirapex®) in accordance with the present invention. Evidence and symptoms of bruxism varied from patient to patient and included soreness of the masseter, temporalis, medial pterygoid and lateral pterygoid muscles, temporomandibular joint tenderness, headache and migraine. The patients were treated with 0.125 to 1.0 mg of pramipexzole one to three times per day for a total of 30 to 90 days. Results were obtained for seven of the nine patients treated. Of these seven patients, six exhibited a reduction or elimination of one or more symptoms or evidence of bruxism, including the reduction or elimination of headache, jaw pain and migraine.

### Example 2

Sixteen patients with symptoms and evidence of bruxism were treated with ropinirole (Requip®) in accordance with the present invention. Evidence and symptoms of bruxism varied from patient to patient and included soreness of the masseter, temporalis, medial pterygoid and lateral pterygoid muscles, temporomandibular joint tenderness, headache and migraine. The patients were treated with 0.25 mg to 3.0 mg of ropinirole one to three times per day for a total of 30 to 90 days. Results were obtained for thirteen of the sixteen patients treated. Of these thirteen patients, eleven exhibited a reduction or elimination of one or more symptoms or evidence of bruxism, including the reduction or elimination of headache, jaw pain and migraine.

### Example 3

Seven patients with symptoms and evidence of bruxism were treated with rotigotine (Neupro®) in accordance with the present invention. Evidence and symptoms of bruxism varied from patient to patient and included soreness of the masseter, temporalis, medial pterygoid and lateral pterygoid muscles, temporomandibular joint tenderness, headache and migraine. The patients were treated with 2.0 mg to 4.0 mg of rotigotine per day for a total of 30 to 60 days. Results were obtained for six of the seven patients treated. Of these six patients, five exhibited a reduction or elimination of one or more symptoms or evidence of bruxism, including the reduction or elimination of headache, jaw pain and migraine.

### Example 4

Two patients with symptoms and evidence of bruxism were treated with rasigiline (Azilect®) in accordance with the present invention. Evidence and symptoms of bruxism varied from patient to patient and included soreness of the masseter, temporalis, medial pterygoid and lateral pterygoid muscles, temporomandibular joint tenderness, headache and migraine. The patients were treated with 0.5 mg to 1.0 mg of rasigiline one time per day for a total of 30 days. Of the two patients treated, both exhibited a reduction or elimination of one or more symptoms or evidence of bruxism, including the reduction or elimination of headache, jaw pain and migraine.

It is to be understood that this invention is not limited to the particular methods, formulations and materials disclosed herein as such methods, formulations and materials may vary somewhat. It is also to be understood that the terminology employed herein is used for the purpose of describing particular embodiments only and is not intended to be limiting since the scope of the present invention will be limited only by the appended claims and equivalents thereof.

All publications and patents mentioned herein are incorporated herein by reference in their entirety for the purpose of describing and disclosing, for example, the materials and methodologies that are described in the publications, which might be used in connection with the presently described invention. The publications discussed above and throughout the text are provided solely for their disclosure prior to the filing date of the present application. Nothing herein is to be construed as an admission that the inventor is not entitled to antedate such disclosure by virtue of prior invention.

The embodiments of the invention in which an exclusive property or privilege is claimed are defined as follows:

1. A method for preventing or treating bruxism, comprising administering to a patient in need of such prevention or treatment a therapeutically effective amount of a dopamine agonist or a pharmaceutically acceptable salt, enantiomer, solvate, hydrate, polymorph or prodrug thereof.

2. The method according to claim 1, wherein the dopamine agonist is a nonergot dopamine agonist.

3. The method according to claim 2 wherein the nonergot dopamine agonist is pramipexole.

4. The method according to claim 2, wherein the nonergot dopamine agonist is ropinirole.

5. The method according to claim 2, wherein the nonergot dopamine agonist is rotigotine.

6. The method according to claim 2, wherein the nonergot dopamine agonist is pardoprunox.

7. The method according to claim 2, wherein the nonergot dopamine agonist is sumanirole.

8. The method according to claim 2, wherein the nonergot dopamine agonist is piribedil.

9. The method according to claim 1 wherein such prevention or treatment reduces or eliminates one or more symptoms, diseases or conditions associated with or resulting from bruxism.

10. The method according to claim 9, wherein the symptom, disease or condition associated with or resulting from bruxism is chronic headache.

11. The method according to claim 9, wherein the symptom, disease or condition associated with or resulting from bruxism is tooth damage.

12. The method according to claim 9, wherein the symptom, disease or condition associated with or resulting from bruxism is temporomandibular disorder.

13. The method according to claim 9, wherein the symptom, disease or condition associated with or resulting from bruxism is migraine headache.

14. The method according to claim 9, wherein the symptom, disease or condition associated with or resulting from bruxism is upper neck pain.

15. The method according to claim 9, wherein the symptom, disease or condition associated with or resulting from bruxism is jaw pain.

16. A method for preventing or treating bruxism, comprising administering to a patient in need of such prevention or treatment a therapeutically effective amount of a momoamine oxidase inhibitor that increases dopaminergic activity or a pharmaceutically acceptable salt, enantiomer, solvate, hydrate, polymorph or prodrug thereof.

17. The method according to claim 16 wherein the monoamine oxidase inhibitor is rasagiline.

18. The method according to claim 16 wherein such prevention or treatment reduces or eliminates one or more symptoms, diseases or conditions associated with or resulting from bruxism.

19. The method according to claim 18, wherein the symptom, disease or condition associated with or resulting from bruxism is chronic headache.

20. The method according to claim 18, wherein the symptom, disease or condition associated with or resulting from bruxism is tooth damage.

21. The method according to claim 18, wherein the symptom, disease or condition associated with or resulting from bruxism is temporomandibular disorder.

22. The method according to claim 18, wherein the symptom, disease or condition associated with or resulting from bruxism is migraine headache.

23. The method according to claim 18, wherein the symptom, disease or condition associated with or resulting from bruxism is upper neck pain.

24. The method according to claim 18, wherein the symptom, disease or condition associated with or resulting from bruxism is jaw pain.

25. A method for preventing or treating bruxism, comprising administering to a patient in need of such prevention or treatment a therapeutically effective amount of a compound that acts as a dopaminergic agent or a pharmaceutically acceptable salt, enantiomer, solvate, hydrate, polymorph or prodrug thereof.
